(19) United States
(12) Patent Application Publication
(10) Pub. No.: US 2012/0143137 A1
(43) Pub. Date: Jun. 7, 2012

(54) INSERTER MECHANISMS

(75) Inventors: Avraham Shekalim, Nesher (IL); Noam Peleg, Gan-Ner (IL)

(73) Assignee: MEDX-SET Ltd., Tirat-Caremel (IL)

(21) Appl. No.: 13/382,968

(22) PCT Filed: Jul. 7, 2010
(86) PCT No.: PCT/IB10/53112

§ 371 Date: Feb. 22, 2012

Related U.S. Application Data
(60) Provisional application No. 61/223,489, filed on Jul. 7, 2009.

(51) Int. Cl. A61B 17/34

(52) U.S. Cl. 604/164.12

(57) ABSTRACT

An Inserter for inserting a needle through the skin of a user that includes: a needle holder block deployed in the inserter housing, the needle holder block displaceable between a cocked position and an insertion position such that when in the cocked position the needle holder block is biased toward the insertion position; a biasing element configured to bias the needle holder block toward the insertion position when the needle holder block is in the cocked position; and a manually operated mechanical controller for controlling displacement of the needle holder block so as to control at least a speed at which the needle holder block is displaced between the cocked position and the insertion position. Also described is an inserter having a mechanism for varying the biasing force of the biasing element so as to affect at least a speed at which said needle holder block is displaced.

### FIELD OF THE INVENTION

The present invention relates to inserter mechanisms for improvement of inserter devices for the insertion of a drug delivery device through the skin of a user, and most preferably, for insertion of a flexible canula which is then left in place for a period of time for continuous or intermittent delivery of a drug into the body.

While inserter devices for the insertion of a flexible canula which is then left in place for a period of time for continuous or intermittent delivery of a drug into the body are well known in the art, the present invention provides certain improvements that increase the usability and user friendliness of such devices.

Therefore, there is a need for improvements to canula insertion devices.

### SUMMARY OF THE INVENTION

The present invention is improvements to canula insertion devices

According to the teachings of the present invention there is provided, an inserter for inserting a needle of a drug delivery device through the skin of a user, the inserter comprising: (a) an inserter housing; (b) a needle holder block deployed in the inserter housing, the needle holder block displaceable between a cocked position and an insertion position such that when in the cocked position the needle holder block is biased toward the insertion position; (c) a biasing element deployed in the inserter hosing and configured to bias the needle holder block toward the insertion position when the needle holder block is in the cocked position; and (d) a manually operated mechanical controller for controlling displacement of the needle holder block between the cocked position and the insertion position so as to control at least a speed at which the needle holder block is displaced between the cocked position and the insertion position.

According to a further teaching of the present invention, the biasing element is a helical spring.

According to a further teaching of the present invention, the manually operated mechanical controller includes at least a first inclined surface against which at least part of the needle holder block bears so as to control at least a speed at which the needle holder block is displaced between the cocked position and the insertion position.

According to a further teaching of the present invention, the needle holder block includes at least one lateral pin projection extending from a side of the needle holder block, the lateral pin projection configured to engage the first inclined surface.

According to a further teaching of the present invention, the first inclined surface is configured to engage the lateral projection pin so as to lock the needle holder block in the cocked position until the manually operated mechanical controller is operated.

According to a further teaching of the present invention, there is also provided a second inclined surface that is deployed facing the first inclined surface such that the lateral projection pin passes between the first and second inclined surfaces as the needle holder block is displaced between the cocked position and the insertion position.

According to a further teaching of the present invention, the manually operated mechanical controller includes a controller spring element configured to provide the manually operated mechanical controller with an opposing biasing force that balances a biasing force of the biasing element.

According to a further teaching of the present invention, the inserter housing includes guide elements configured to guide the needle holder block as the needle holder block is displaced between the cocked position and the insertion position.

According to a further teaching of the present invention, there is also provided a needle retraction mechanism configured to retract the needle holding block away from the insertion position once the needle holder clock reaches the insertion position.

There is also provided according to the teachings of the present invention, an inserter for inserting a needle of a drug delivery device through the skin of a user, the inserter comprising: (a) an inserter housing; (b) a needle holder block deployed in the inserter housing, the needle holder block displaceable between a cocked position and an insertion position such that when in the cocked position the needle holder block is biased toward the insertion position; (c) a biasing element deployed in the inserter hosing and configured to bias the needle holder block toward the insertion position when the needle holder block is in the cocked position; (d) a release element for releasing the needle holder block enabling the displacement between the cocked position and the insertion position; and (e) a mechanism for varying the biasing force of the biasing element so as to affect at least a speed at which the needle holder block is displaced between the cocked position and the insertion position.

According to a further teaching of the present invention, the mechanism for varying the biasing force of the biasing element includes a handle that treadedly engages the inserter housing such that rotation of the handle varies a position within the inserter housing of a base element to which the biasing element is attached and the varying of the position of the base element varies the biasing force of the biasing element.

According to a further teaching of the present invention, an initial position of the base element is such that the biasing force of the biasing element is at a maximum state when the needle holder block is in the cocked position and varying the biasing force of the biasing element is decreasing the biasing force of the biasing element.

### BRIEF DESCRIPTION OF THE DRAWINGS

The invention is herein described, by way of example only, with reference to the accompanying drawings, wherein:

FIG. 1 is a cross-sectional view of a first embodiment of an inserter device having a manually operated mechanical controller for controlling displacement of a needle holder block constructed and operational according to the teachings of the present invention, shown here in a cocked position;

FIG. 2 is an isometric view of the manually operated mechanical controller of FIG. 1;

FIG. 3 is a cross-sectional view of the controller of FIG. 1, shown in a partially inserted position;

FIG. 4 is a cross-sectional view of the controller of FIG. 1, shown in a fully inserted position;

FIG. 5 is an isometric cut-away view of the inserter of FIG. 1;

FIG. 6 is a cross-sectional view of a second embodiment of an inserter device having a manually operated mechanical controller for controlling displacement of a needle holder block constructed and operational according to the teachings of the present invention, shown here in a cocked position;

FIG. 7 is a cross-sectional view of the controller of FIG. 6, shown in a partially inserted position;

FIG. 8 is a cross-sectional view of a variant of the controller of FIG. 6, shown in a cocked position; and

FIG. 9 is a cross-sectional view of an inserter having a mechanism for varying the biasing force of the biasing element constructed and operational according to the teachings of the present invention.

### DESCRIPTION OF THE PREFERRED EMBODIMENTS

The present invention is improvements to canula insertion devices

The principles and operation of improvements to canula insertion devices according to the present invention may be better understood with reference to the drawings and the accompanying description.

By way of introduction, it has been found that some users have an aversion to “firing” a needle into their body by sudden release of spring by a trigger. To address this issue, the second aspect of the present invention provides a manually operated mechanical controller for controlling at least the speed and force of the motion of the inserter needle to allow slow or rapid insertion.

The mechanism is triggered by motion of the trigger or release element to release a primary spring which forces the needle holder block carrying the inserter needle downwards into the skin. The motion of the needle holder block is controlled by the speed at which the trigger is depressed into the inserter housing.

At the end of the insertion motion, a retraction mechanism may be engaged so as to withdraw the needle holder block together with the needle while the canula remains inserted.

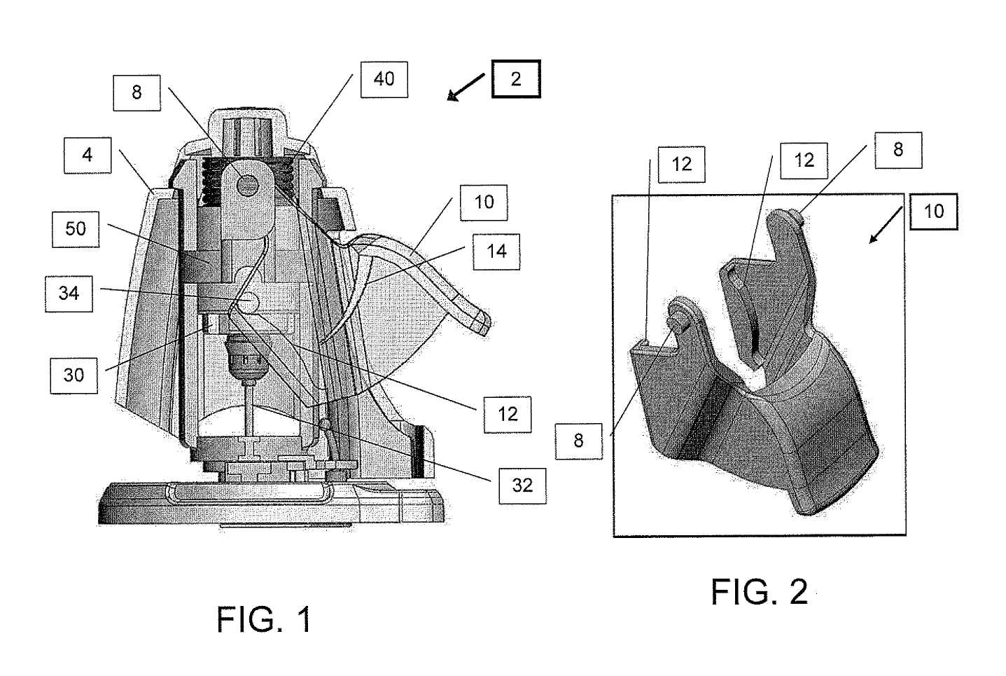

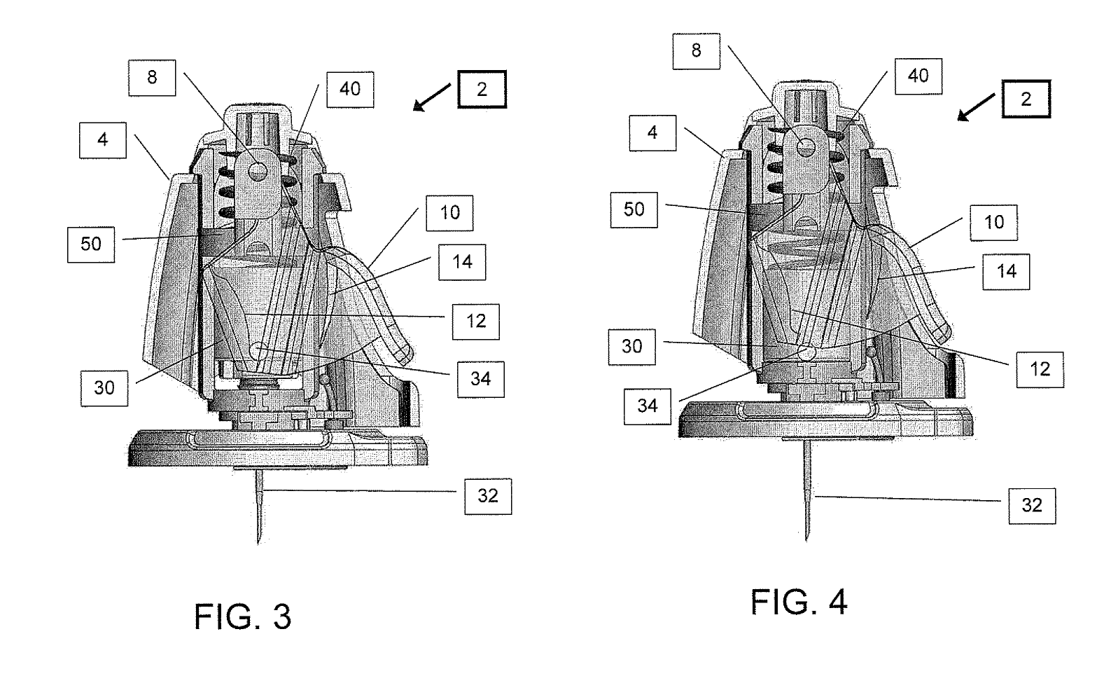

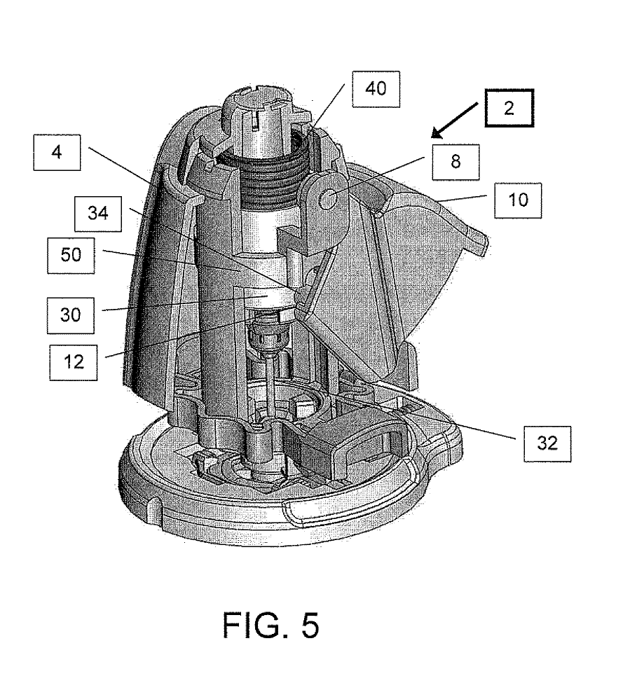

Referring now to the drawings, structurally, in the non-limiting implementation of an insertion device 2 shown here in FIGS. 1-5, the trigger or release element 10 is attached to the inserter housing 4 and rotates about pins 8. Associated with the release element 10 is an inclined surface 12 against which part of the needle holder block 30 bears, under bias of the primary insertion spring 40. Progressive manual depression of the release element 10, resisted by an opposing spring (here implemented as an integrally molded spring element 14), successively displaces the inclined surface(s) 12 to allow the needle holder block 30 and needle 32 to advance under action of the primary spring 40. In the example shown here, two inclined surfaces 12 are provided on opposite sides of the needle holder block 30, thereby providing symmetrical support. Lateral pin projections 34 extending from the two sides of the needle holder block 30 engage the inclined surfaces 12.

The inclined surfaces 12 may be profiled, for example as shown, to provide an initial self-locking state and/or to provide any other actuator-to-needle-displacement profile desired. In the non-limiting example illustrated here, a final part of the needle motion occurs after the pins 34 have cleared the inclined surfaces, thereby providing a positive “click” engagement of the canula connector in the base unit (see FIG. 4).

Although not shown here, this mechanical controller mechanism can be combined with a retraction mechanism.

Optionally, although not necessarily, an additional sliding engagement may be provided to define the path of motion of the needle holder block. Such as the embodiment illustrated in FIGS. 1-5, which includes an internal chassis 50 in which the needle holder block is displaced.

Most preferably, the balance of forces between the primary spring 40 and the opposing spring 14 together with the angle of the inclined surfaces 12 are chosen so as to allow stopping of the needle 32 at any point along at least part, and preferably along the majority, of the path of motion of the needle. This allows the user to feel he or she has full control over the insertion process. At the same time, the structure allows rapid motion of the actuator so that the needle will be inserted at high velocity, thereby achieving substantially painless insertion similar to the freely released spring driven motion of prior art embodiments.

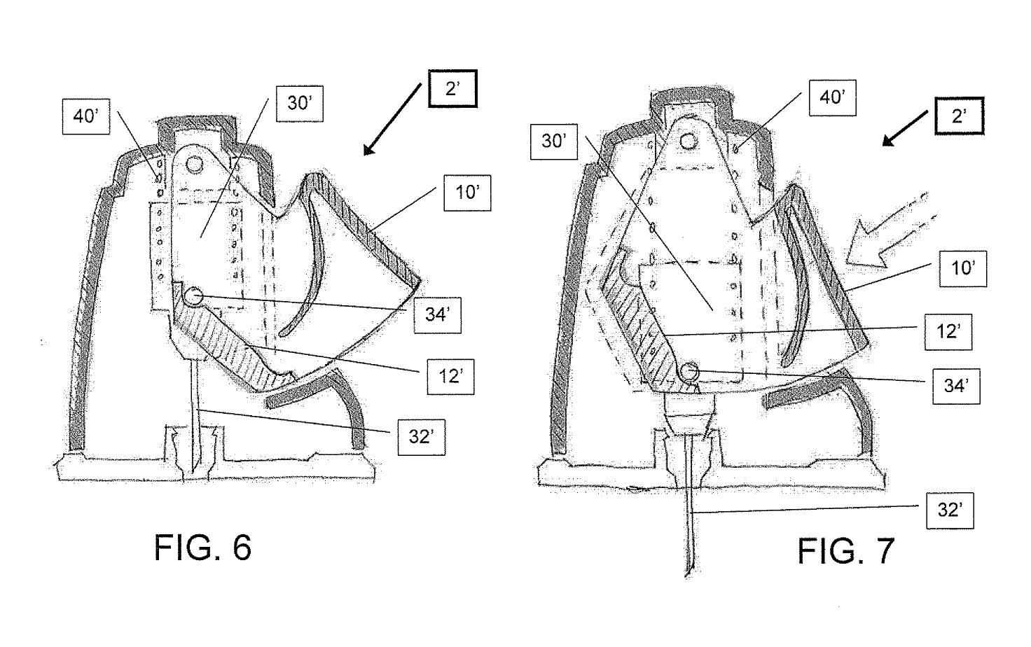

The embodiments illustrated in FIGS. 6-8 have non-guided needle holder blocks, which is essentially the only difference between the embodiment of FIGS. 1-5 and the embodiment of FIGS. 6 and 7.

As in the embodiment above, the inserter device of FIGS. 6 and 7 includes an inserter housing 2′ in which are deployed a needle holder block 30′ that is displaceable between a cocked position (FIG. 6) and an insertion position (FIG. 7 illustrates a partial insertion position).

The needle holder block 30′ is biased, and ultimately displaced by, spring element 40′ with such displacement being controlled by a manually operated mechanical controller 14′ for controlling at least the speed and force of the motion of the inserter needle. As described above, the manual control of the displacement of the needle holder block is influenced by the interaction between the lateral pin projections 34′ that extend from the sides of the needle holder block 30′ and interact with the inclined surface 12′.

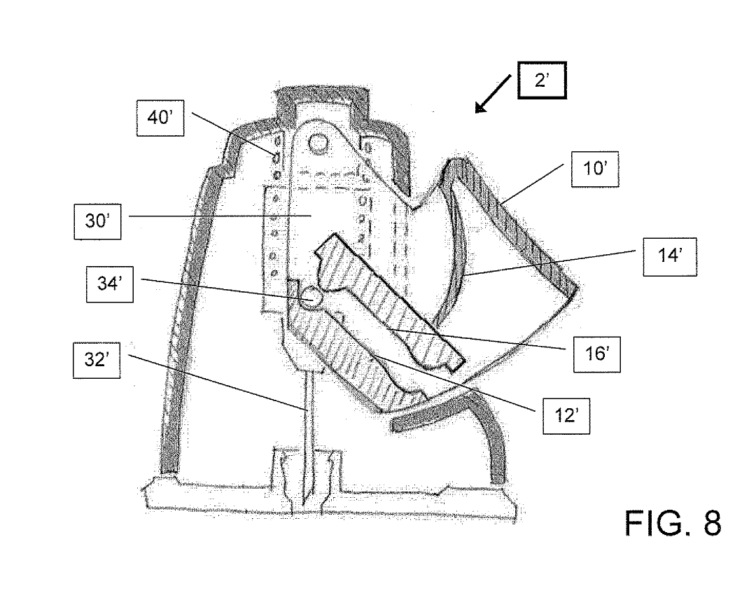

The embodiment of the present invention illustrated in FIG. 8, includes all of the elements and operates the same as the embodiment of FIGS. 6 and 7 above, with the addition of a second set of inclined surfaces 16′. Inclined surface 16′ is deployed opposite and parallel to the first inclined surface 12′, thereby providing a channel through which the lateral pin projections 34′ travel. This serves to stabilize the needle holder block within the inserter housing as it is displaced between the cocked position and the insertion position. As in the embodiments described above, the progressive manual depression of the release element 10′ is resisted by an opposing spring illustrated here as an integrally molded spring element 14′. It will be appreciated that numerous other configurations for stabilizing the needle holder block within the inserter housing as it is displaced are possible and are within the scope of the present invention.

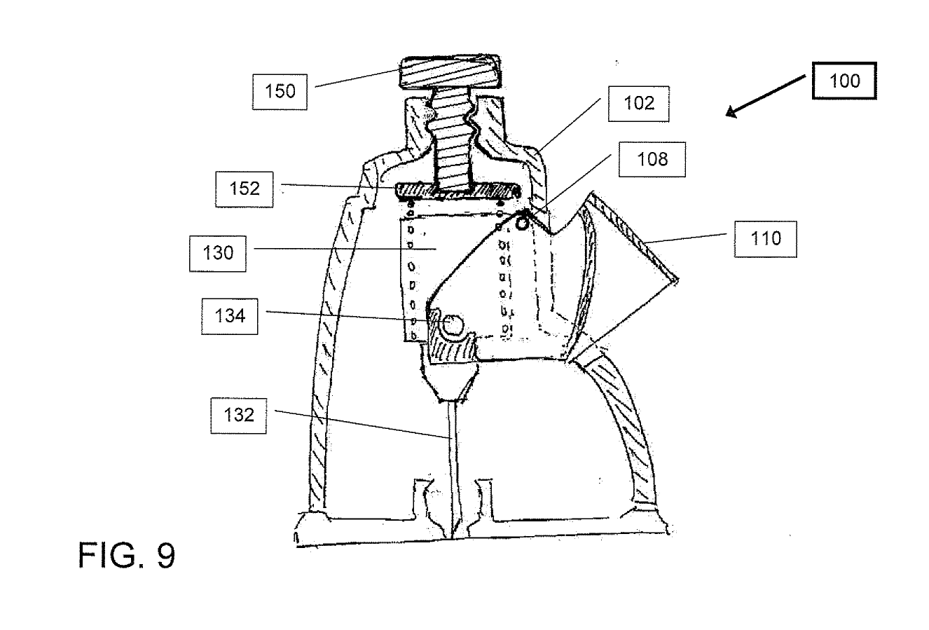

FIG. 9 illustrates a third embodiment of an inserter device 100 for inserting the needle 132 of a drug delivery device through the skin of a user according to the teachings of the present invention.

Inserter 100 includes an inserter housing 102 in which is deployed a needle holder block 130. As in the embodiments above, the needle holder block 130 is displaceable between a cocked position (as shown in FIG. 9) and an insertion position. When in the cocked position the needle holder block 130 is biased toward the insertion position.

A biasing element, shown here as a helical spring 140, is deployed in the inserter hosing and configured to bias the needle holder block 130 toward the insertion position when the needle holder block 130 is in the cocked position.

Release element or trigger 110 is attached to the inserter housing 102 and rotates about pins 108. The release element configured to hold the needle holder block 130 in the cocked position until it is released thereby enabling the displacement of the needle holder block 130 between the cocked position and the insertion position.

Unique to this embodiment is a mechanism for varying the biasing force of the biasing element 140 so as to affect at least a speed at which the needle holder block 130 is displaced between the cocked position and the insertion position.

As illustrated here, the mechanism for varying the biasing force of the biasing element 140, illustrated as a helical spring, includes a handle 150 that threadingly engages the inserter housing 102 such that rotation of the handle 150 varies a position within the inserter housing 102 of a spring base element 152 to which the biasing element 140 is attached. Therefore, varying of the position of the spring base element 152 varies the biasing force of the biasing element 140.

Preferably, but not necessarily, an initial position of the spring base element 152 is such that the biasing force of the biasing element 140 is at a maximum state when the needle holder block is in the cocked position. From this deployment, varying the biasing force of the biasing element 140 is limited to decreasing the biasing force of the biasing element 140.

In one variant of the embodiment of FIG. 9 not illustrated, the mechanism for varying the biasing force of the biasing element includes a plurality of spring elements having varied force properties deployed within the inserter housing. Each of the spring elements of the plurality is configured for selective engagement to the needle holding block such that selective engagement of any one or combination of more than one spring elements varies the biasing force.

In another variant of the embodiment of FIG. 9 not illustrated, the mechanism for varying the biasing force of the biasing element includes at least one damping element. The damping elements may be either hydraulic or pneumatic in nature or a mechanical friction element used as a damper.

It will be appreciated that the damping element may be adjustable so as to allow for varying the damping properties of the damping element, similar to and adjustable shock absorber as is know in the automotive arts.

Alternatively, the damping element may be configured as a plurality of damping elements being selectively engagable to the needle holding block such that selective engagement of any one or combination of more than one of the damping elements varies the biasing force of the biasing element.

It will be appreciated that the above descriptions are intended only to serve as examples and that many other embodiments are possible within the scope of the present invention as defined in the appended claims.

What is claimed is:

1. An inserter for inserting a needle of a drug delivery device through the skin of a user, the inserter comprising:
(a) an inserter housing;
(b) a needle holder block deployed in said inserter housing, said needle holder block displaceable between a cocked position and an insertion position such that when in said cocked position said needle holder block is biased toward said insertion position;
(c) a biasing element deployed in said inserter hosing and configured to bias said needle holder block toward said insertion position when said needle holder block is in said cocked position; and
(d) a manually operated mechanical controller associated with said housing and configured for controlling displacement of said needle holder block between said cocked position and said insertion position so as to control at least a speed at which said needle holder block is displaced between said cocked position and said insertion position.

2. The inserter of claim 1, wherein said biasing element is a helical spring.

3. The inserter of claim 1, wherein said manually operated mechanical controller includes at least a first inclined surface against which at least part of said needle holder block bears so as to control at least a speed at which said needle holder block is displaced between said cocked position and said insertion position.

4. The inserter of claim 3, wherein said needle holder block includes at least one lateral pin projection extending from a side of said needle holder block, said lateral pin projection configured to engage said first inclined surface.

5. The inserter of claim 4, wherein said first inclined surface is configured to engage said lateral projection pin so as to lock said needle holder block in said cocked position until said manually operated mechanical controller is operated.

6. The inserter of claim 4, further including a second inclined surface that is deployed facing said first inclined surface such that said lateral projection pin passes between said first and second inclined surfaces as said needle holder block is displaced between said cocked position and said insertion position.

7. The inserter of claim 1, wherein said manually operated mechanical controller includes a controller spring element configured to provide said manually operated mechanical controller with an opposing biasing force that balances a biasing force of said biasing element.

8. The inserter of claim 1, wherein said inserter housing includes guide elements configured to guide said needle holder block as said needle holder block is displaced between said cocked position and said insertion position.

9. The inserter of claim 1, further including a needle retraction mechanism configured to retract said needle holding block away from said insertion position once said needle holder clock reaches said insertion position.

10. An inserter for inserting a needle of a drug delivery device through the skin of a user, the inserter comprising:
(a) an inserter housing;
(b) a needle holder block deployed in said inserter housing, said needle holder block displaceable between a cocked position and an insertion position such that when in said cocked position said needle holder block is biased toward said insertion position;
(c) a biasing element deployed in said inserter hosing and configured to bias said needle holder block toward said insertion position when said needle holder block is in said cocked position;
(d) a release element associated with said housing and configured for releasing said needle holder block enabling said displacement between said cocked position and said insertion position; and
(e) a mechanism for varying the biasing force of said biasing element so as to affect at least a speed at which said needle holder block is displaced between said cocked position and said insertion position.

11. The inserter of claim 10, wherein said mechanism for varying the biasing force of said biasing element includes a handle that treadedly engages said inserter housing such that rotation of said handle varies a position within said inserter housing of a base element to which said biasing element is attached and said varying of said position of said base element varies said biasing force of said biasing element.

12. The inserter of claim 11, wherein an initial position of said base element is such that said biasing force of said biasing element is at a maximum state when said needle holder block is in said cocked position and varying the biasing force of said biasing element is decreasing said biasing force of said biasing element.
